(19) United States
(12) Patent Application Publication
(10) Pub. No.: US 2005/0175654 A1
(43) Pub. Date: Aug. 11, 2005

(54) Fiber assisted emulsion system

(76) Inventors: Dean M. Willberg, Moscow (RU); Curt Dacar, Bakersfield, CA (US)

Correspondence Address: SCHLUMBERGER TECHNOLOGY CORPORATION, IP DEPT., WELL STIMULATION, 110 SCHLUMBERGER DRIVE, MD1, SUGAR LAND, TX, 77478, US

(21) Appl. No.: 11/105,930

(22) Filed: Apr. 14, 2005

Related U.S. Application Data
(62) Division of application No. 10/248,675, filed on Feb. 7, 2003.
(60) Provisional application No. 60/412,430, filed on Sep. 20, 2002.

(51) Int. Cl. A61K 7/00

(52) U.S. Cl. 424/401

(57) ABSTRACT

Emulsions, either water-in-oil or oil-in-water, may be formed by combining an aqueous component, a non-aqueous component and a surfactant in combination with fibers. The fibers decrease the time and energy required to form the emulsion and, in some cases, allow emulsion formation that would not be possible with the use of such fibers.

### BACKGROUND OF INVENTION

The present invention relates generally to fluids useful in treating a subterranean formation. More specifically, the invention is fiber assisted emulsion system.

The Fiber Assisted Transport (FAT) fluid technology is becoming more and more accepted in the oilfield services industry for fracturing oil bearing formations, particularly diatomite formations. One fluid that is currently in use is a mixture of 75 lbm/1000 gal polyester staple fiber in 30 lbm/1000 gal guar based fluid. This technology reduces costs and increases the net profit generate from fracturing treatments, primarily by reducing the proppant required to maintain production at acceptable levels.

An emulsion is generally defined as a mixture of particles of one liquid with a second liquid. Typically, one liquid is aqueous, while the second is non-aqueous (i.e., insoluble in the aqueous liquid). Therefore, two common types of emulsions include “oil-in-water,” in which the aqueous phase is continuous and “water-in-oil” in which the non-aqueous phase is continuous.

In most cases, simply combining an aqueous liquid with a non-aqueous liquid, even after sufficient mixing, will not promote emulsion formation or, alternatively, will produce unstable, short-lived emulsions. An emulsifying agent or surfactant is also required to enable the emulsion to form and remain relatively stable.

The use of particles to stabilize emulsions is not new, margarine being a good example of a prior discovery. With regard to-the oilfield industry, U.S. Pat. No. 5,294,353 (Dill) describes the use solid particles to stabilize emulsions in oil-based drilling fluids. Silica flour has also been shown to stabilize water-in-oil emulsions used in acidizing and acid fracturing. In general, the particulates used in these examples are quite small, and with the exception of possibly functioning as fluid loss additives, they would not be useful for well stimulation applications. In fact, these particles would likely damage the permeability of proppant packs, screens or even of the rock matrix without added benefit.

In order to reduce costs and minimize polymer damage in low-pressure oil reservoirs, it would be beneficial to develop an emulsion system that used locally produced crude oil (also known as lease oil) as the base fluid.

### SUMMARY OF INVENTION

The present invention provides a novel method of producing both oil-in-water (“o/w”) and water-in-oil (“w/o”) emulsions and hyperemulsions through the use of fibers. The addition of fibers during the preparation of emulsions decreases the time required and the energy required (i.e., mixing force or agitation) to form the emulsion.

In a method of the present invention, fibers are mixed with the aqueous phase, the oil phase, and an appropriate surfactant. The components are then agitated and an emulsion is formed. After emulsification, the fibers may be removed by filtration prior to use of the emulsion.

The addition of a hydrophilic fiber and appropriate surfactant greatly accelerates the rate of formation of water-external emulsions while the addition of hydrophobic fibers and appropriate surfactant accelerates the formation of oil-external emulsions. In many cases the particular emulsion could not be readily formed without the addition of fibers and in all cases, the time and energy required to generate the emulsion was reduced with the addition of fibers.

Emulsions prepared by the methods described herein are typically relatively stable (i.e., many days at room temperature) even after the fibers have been filtered out or otherwise removed from the mixture. Furthermore, by using fibers, an emulsion with as little as 3-4% external aqueous phase may be formed, using the same surfactant as that used in current commercial emulsion systems. Typically, commercially available emulsion systems will invert, that is the dispersed phase becomes the continuous phase and vice versa, dramatically losing viscosity, if the aqueous phase drops below 28%. That means that the use of fibers greatly extends the range of emulsion stability.

### BRIEF DESCRIPTION OF DRAWINGS

FIG. 1 is a rheogram of an oil-in-water emulsions formed in diesel oil using two different concentrations of a mixture of ethoxylated alcohols as surfactant and polyester fibers.

FIG. 2 is a rheogram of an oil-in-water emulsion formed in diesel oil using two different concentrations of a cationic surfactant and polyester fibers.

FIG. 3 is a rheogram of an oil-in-water emulsions formed in diesel oil using two different concentrations of a sodium lauryl surfactant and polyester fibers.

FIG. 4 is a rheogram of an oil-in-water emulsions formed in crude oil using a cationic surfactant and polyester fibers.

FIG. 5 is a rheogram of an oil-in-water emulsion as in FIG. 4 with a reduced quantity of surfactant.

FIG. 6 is a rheogram of an oil-in-water emulsion as for FIG. 5 with a lower fiber loading.

FIG. 7 is a rheogram of an emulsion as for FIG. 6 without fibers.

FIG. 8 is a rheogram of an emulsion similar to the one tested for FIG. 6 but with another crude oil.

FIG. 9 is a rheogram of oil-in-water emulsions formed in crude oil using two a mixture of ethoxylated alcohols as surfactants and polyester fibers.

### DETAILED DESCRIPTION

The emulsions and methods of the present invention may use any suitable starting components or materials. Typically, the components necessary to prepare an emulsion in accordance with the present invention include an aqueous component or phase, a non-aqueous component or phase, an emulsifying agent or surfactant and fibers.

In a preferred embodiment of the invention, the aqueous component is a brine. Such brine may contain any suitable amount of salt, as well as other elements or compounds. Particularly, brines commonly found in oilfield locations or used in oilfield applications are preferred. Other suitable aqueous components include polymers. For instance, guars, modified guars, polyacrylamide polymers and copolymers, state soluble modified cellulosic polymers, such as hydroxyl ethyl cellulose (“HEC”), or xanthan. Where the aqueous component of the emulsion is a polymer, it may be beneficial to crosslink the aqueous component.

The non-aqueous component of the present invention may be any suitable liquid or compound. In a preferred embodiment, the non-aqueous component is selected from diesel, kerosene, mineral oil, vegetable oil or crude oil.

Surfactants suitable for forming o/w emulsions include ethoxylated alcohols, quaternary amines, anionic surfactants and sodium lauryl sulfonate. The Hydrophilic Lipophilic Balance and Phase Inversion Temperature approach may be useful in determining the applicability of certain surfactants for use in the present invention. By judicious selection of surfactant species and concentration, an emulsion can be formed that will break under bottomhole conditions.

Fibers useful in the present invention are typically non-symmetrical with at least one dimension in the range of approximately 2-100 microns and a second dimension in the range of approximately 50 microns or greater. Depending on the specific type of emulsion to be formed (i.e., o/w or w/o) the fibers may be hydrophobic or hydrophilic. For use in the preparation of o/w emulsions, hydrophilic fibers are preferred and for the preparation of w/o emulsions, hydrophobic fibers are preferred. In a preferred embodiment, the fibers used are selected from the group consisting of novoloids, aramids, glasses, polyethylene terepthalates and polyamides. The fibers of the present invention may further be dispersible in the aqueous component. A particularly preferred fiber is polyester, which readily disperses in crude oil and will suspend sand. The fibers used to form the emulsions of the present invention need not remain in the emulsion after formation, but rather may be removed, such as by filtration, after formation. The emulsion will remain stable after the fibers have been removed.

In addition to the aqueous component, non-aqueous component, surfactant and fibers, the emulsion may contain any number of additional components, as required for a specific application. For instance, a viscosity increasing agent may be included for applications where a more viscous emulsion is required. Preferably, the viscosity increasing agent is a polymer which is soluble in the aqueous phase. More preferably the viscosity increasing agent is a guar, modified guar, polyacrylamide polymer, polyacrylamide copolymer, HEC or xanthan.

Other additional components may include, for instance, reactive species. These reactive species may be any suitable species required for the function of the fracturing fluid that do not interfere with the formation of the emulsion. For instance, appropriate clay stabilizers or biocides are reactive species useful in the practice of the present invention. In a preferred embodiment, the reactive species is a crosslinking agent. More preferably, the crosslinker is chosen from the following: boric acid, sodium borate, a titanium complex, a zirconium complex or a dialdehyde. In yet another preferred embodiment, the reactive species is a cement retarding agent. Alternatively, the reactive species may also be a pH modifier.

A particulate material may also be included in the emulsion. In a preferred embodiment the particulate material is a proppant. More preferably, the particulate material comprises sand or ceramic particles.

In preparing emulsions in accordance with the present invention, it should be understood that the order of addition of the various components may be varied as needed. For instance, the aqueous component may be combined with the fibers and surfactant prior to combination with the non-aqueous component. Similarly, the fibers and surfactant may be combined with the non-aqueous component prior to combination with the aqueous component. In addition, any suitable mixing process may be used to combine the components. For instance, a continuous mix manner or a batch-process manner of mixing may be used.

Fracturing Applications

Of particular interest is the use of the emulsions of the present invention in oilfield applications. In particular, both o/w and w/o emulsions are useful for fracturing applications, although it should be understood that use of these emulsions is not limited to fracturing. Besides their role in emulsion formation, the fibers also assist in proppant transport and/or in proppant flowback control. These emulsions have adequate viscosity for fracture width creation and proppant transport. Certain, formulations are capable of producing emulsions which are stable at temperatures greater than 250° F.

During fracturing operations, the emulsion may be prepared using any suitable-method. In one embodiment, the components of the emulsion may be combined in the wellbore or immediately prior to entering the wellbore. In such a case, the emulsion would be formed in the wellbore itself. Where required, suitable agitation may be provided in the wellbore to form the emulsion. The emulsion may also be formed downhole. For instance, the individual emulsion components may be pumped or placed downhole prior to mixing or agitation. In a preferred embodiment, a downhole assembly, mixer, jetting device or nozzle may provide suitable agitation downhole.

Diesel and Mineral Oil Examples

The following examples were conducted using a simplified process of generating many water-in-oil (w/o) and oil-in-water (o/w) emulsions. In this method an appropriate fiber/surfactant combination is added to a vessel containing both the aqueous and oil phases. The fluid in the vessel is then agitated. The level of agitation mixing time, mixing intensity, or both required to form the emulsion is less than required to form the emulsion without the fibers present. Indeed, emulsions could not be formed without the addition of fibers in a number of the formulations studied. The process works over a wide range of oil, water and stabilizing surfactant concentrations. Furthermore, a number of both w/o and o/w formulations were prepared that are stable for hours to days after the fibers are filtered out from the emulsion.

In the majority of tests discussed below the fluids were prepared in 1000 mL plastic tri-corner beakers. Unless otherwise stated mixing was performed with a 3-inch diameter 3-blade propeller rotated at 900 rpm by an overhead mixer. Typical formulations used 100 mL of oil, 5-20 mL of the water phase, and additives. In certain cases, a 200 mL batch would be prepared to determine if there were any volumetric effects in the preparation of the emulsion.

Oil in Water Formulations

One formulation studied and used for demonstration purposes contain the following:

[t1]

| | | |
|---|---|---|
| Mineral Oil | 100 | mL |
| A solution of 3 wt % potassium choloride in water | 10 | mL |
| Surfactants (HLB˜3) | 0.15 | mL |
| Polyester fiber | 0.90 | g |

This mixture was agitated with a 3″ 3-blade propeller rotated at 900 rpm by an overhead mixer. Without the fibers the viscous emulsion did not form even after 5 minute of mixing. With the fibers the emulsion formed in under 60 seconds. This formulation produced a 9% external phase emulsion.

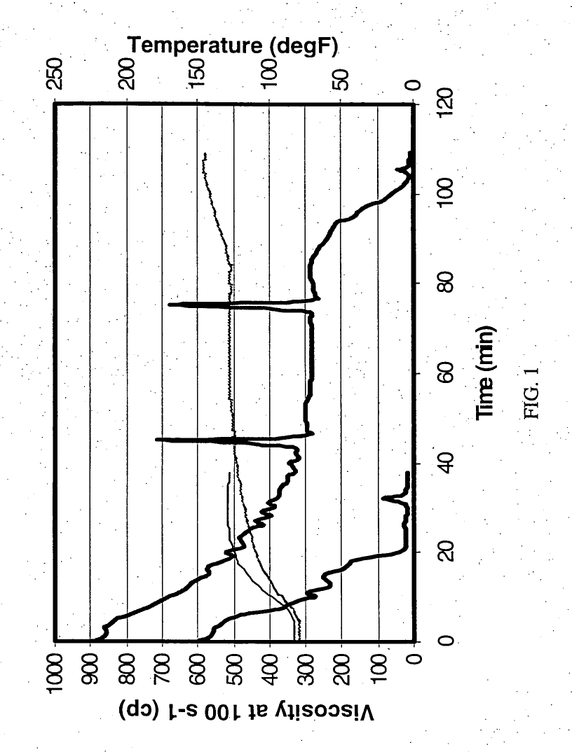

FIGS. 1-3 show the viscosity over time of formulations prepared with different surfactants, at different concentrations. The temperature was adjusted to simulate oilfield conditions, as shown with the hairlines curves. The peaks are due to shear-rates ramps.

FIG. 1 show the effect of surfactant concentration on the stability of the emulsion. The emulsion in this example comprises 100 ml of diesel oil, 10 ml of KCl brine at 3% and 0.90 g of polyester fibers. The surfactant is a mixture of ethoxylated alcohol, at a concentration of 6.8 ml per liter (black curve) or 9.0 ml per liter (grey curve).

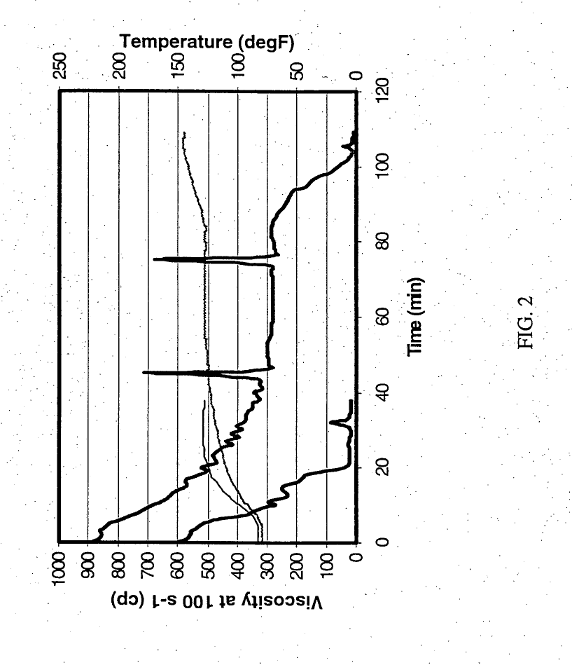

In FIG. 2, the emulsion comprises 100 ml of diesel oil, 10 ml of KCl brine at 3% and 0.90 g of polyester fibers. The surfactant is cationic, at a concentration of 0.9 ml per liter (black curve) and 1.8 ml per liter (grey curve).

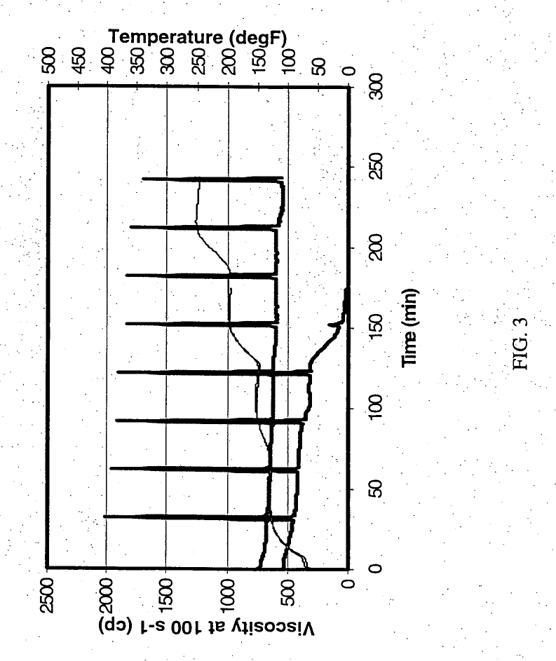

In FIG. 3, the emulsion comprises 100 ml of diesel oil, 10 ml of KCl brine at 3% and 0.90 g of polyester fibers. The surfactant is a sodium lauryl sulfonate, at a concentration of 0.45 ml per liter (black curve) and 2.7 per liter (grey curve).

Fibers with hydrophilic surfaces typically perform better than those with hydrophobic surfaces for forming o/w emulsions. Several different types of fibers have been shown to be effective at forming and stabilizing emulsions, including, polyesters (i.e., PET), polyamides, novoloids, acramids, glasses, and spun limestone fibers that have, or have been treated to have, hydrophilic surfaces.

The advantage of fibers to assist in the formation of the emulsion is readily demonstrated in the following example:

[t2]

| Base Fluid: | |
|---|---|
| Diesel Oil | 100 mL |
| A solution of 3 wt % potassium chloride in water | 5 mL |
| Cationic emulsifier Surfactant for o/w emulsion | 0.10 mL |

In four separate tests the above mixture was agitated with a 3-inch 3-blade propeller rotated at 900 rpm by an overhead mixer. In each test a different quantity of polyester fiber was added. The results of these tests are summarized in the table, below:

[t3]

| Fibers Mass | |
|---|---|
| (g) | Time to Form Fully Developed Emulsion |
| 0.25 | The fibers clumped together in bundles but no emulsion |
| | formed within 5 min of mixing. |
| 0.50 | Emulsion formed in 90 sec. |
| 1.00 | Emulsion formed in 40-60 sec. |
| 2.00 | Emulsion formed in ˜40 sec. |

These tests show that increased fiber quantity decreases emulsion formation time in certain formulations.

Water-In-Oil Formulations

By choosing a hydrophobic fiber, and by using a suitable surfactant fibers may also be used to assist in the formation of water-in-oil emulsions. The following examples demonstate this:

[t4]

| | |
|---|---|
| A solution of 3 wt % potassium chloride in water | 100 mL |
| Mineral Oil | 5 mL |
| Surfactant blend formulated to form w/o emulsions | 0.15 mL |
| Polypropylene Fiber (2.2 denier) | 0.90 g |

This mixture was agitated with a 3- inch 3-blade propeller rotated at 900 rpm by an overhead mixer. Alternatively the mixture can be shaken vigorously in a bottle. Without the fibers the viscous emulsion did not form even after five minutes of mixing in the above equipment. A hyper-emulsion was formed without the fiber only after extended mixing with a high shear rate Silverson mixer. Fibers with hydrophobic surfaces, such as polypropylene work best for this process. In one test an emulsion was formed after 2-3 of mixing when hydrophilic polyester fibers were used. It is possible that in that treatment the hydrophilic finish on the fiber was stripped off, leaving a hydrophobic surface.

Crude Oil Examples

The following examples were prepared using crude oil as the non-aqueous component. Emulsions consistently formed in the crude oils tested if: 1) A polymer composition with greater than 10 lbm/ 1000 guar was used for the aqueous phase, 2) the water phase was greater than about 10-17% of the total emulsion volume, and 3) the fibers, or a small fraction of the fibers, were wetted with the aqueous phase prior to the introduction of the crude oil.

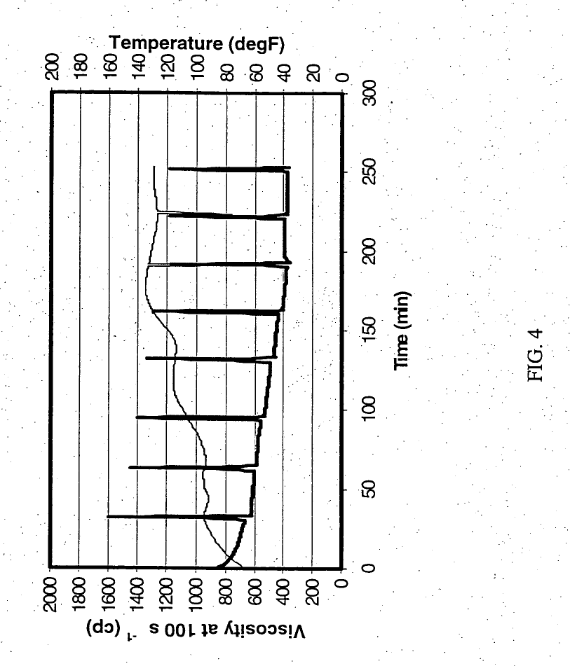

FIGS. 4-9 show the viscosity over time of formulations prepared with different surfactants. The temperature was adjusted to simulate oilfield conditions, as shown with the hairlines curves. The peaks are due to shear-rates ramps.

In FIG. 4, the emulsion comprises 200 ml of Belridge crude oil, 40 ml of a water-based fracturing fluid (loaded with guar at 15 gal/1000 gal of base fluid) and 1.8 g of polyester fibers and 0.4 ml of a cationic surfactant.

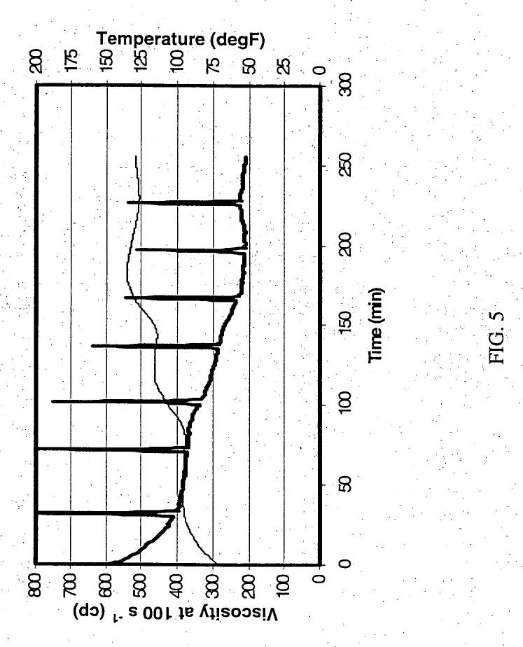

FIG. 5 is identical to FIG. 4, except that the quantity of surfactant was reduced to 0.2 ml.

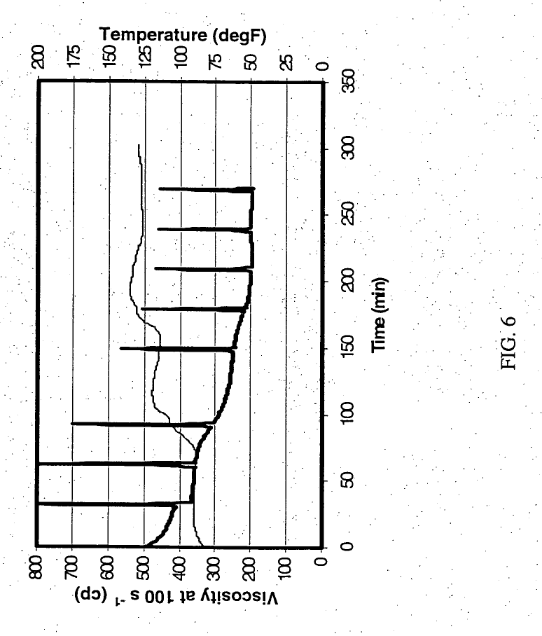

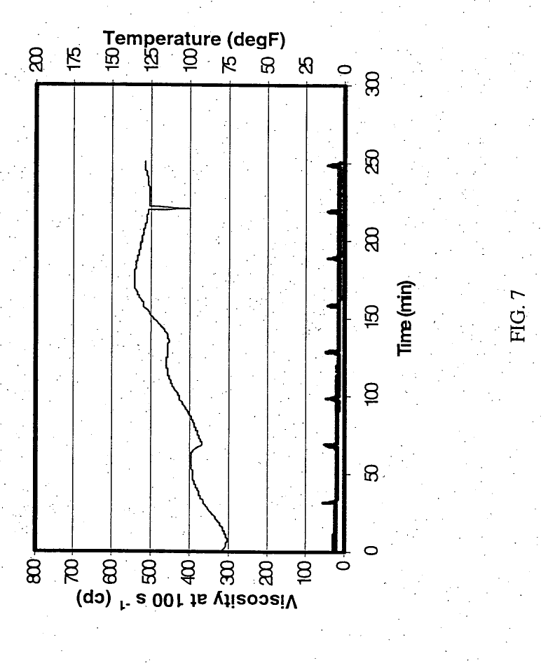

FIG. 7 shows a control test with the same fluid as the one tested FIG. 6, this time in absence of fibers.

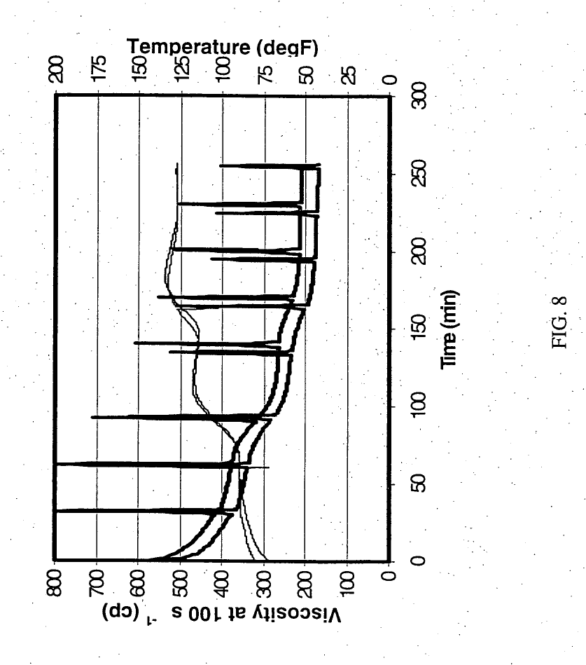

The same measurements were performed for FIG. 8, with another crude oil but otherwise under the same conditions as for FIG. 6. The rheogram in black was measured on a fluid in which the fibers had been removed by filtering.

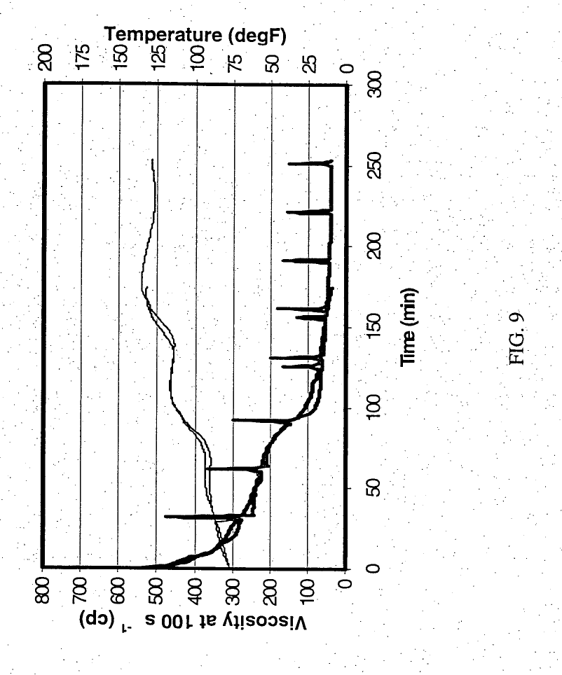

FIG. 9 is a rheogram obtained with an emulsion formed using Belridge crude oil (200 ml), 40 ml of a water-based fracturing fluid base (water at 15 lbs/1000 gal of guar) and 1 ml (or 4.2 gal/1000 gal) of a mixture of ethoxylated alcohols as the surfactant. This emulsion was prepared with a low fiber loading (6.9 lbm/1000 gal of the total emulsion). The fluid readily breaks at about 120° F.

These examples demonstrate that the use of fibers promotes emulsion formation and emulsion. It should be understood the preceding examples are for demonstration purposes and are not intended to show every possible combination of components useful in the present invention. Combinations not specifically disclosed in the examples may still be within the spirit and scope of the foregoing disclosure and the following claims.

With FIG. 6, the fluid is the same as in FIG. 5, except that the quantity of fibers has been reduced to 0.2 g, thus corresponding to a low fiber loading equivalent to 6.9 lbm/1000 gal (compared to 62.5 lbm/1000 gal for FIGS. 4 and 5).

1. A method for producing an emulsion comprising the steps of:
(a) providing a non-aqueous component;
(b) providing an aqueous component;
(c) providing a surfactant;
(d) providing fibers, and
(e) wherein said non-aqueous component, aqueous component, surfactant and fibers are combined to form the emulsion.

2. The method of claim 1, wherein said fibers have a first dimension in the range of approximately 2-100 microns and a second dimension in the range of approximately 50 microns or greater.

3. The method of claim 1, comprising the further step (f) of removing said fibers from the emulsion.

4. The method of claim 3, wherein said fibers are removed by filtration.

5. The method of claim 1, wherein said fibers are hydrophilic.

6. The method of claim 5, wherein the fibers and surfactant are combined with the aqueous component prior to combination with the non-aqueous component.

7. The method of claim 1, wherein said fibers are dispersible in the aqueous component.

8. The method of claim 7, wherein said fibers are selected from the group comprising, polyesters, polyamides, novoloids, aramids, glasses, polyethylene terepthalates or polyamides.

9. The method of claim 1, wherein said fibers are hydrophobic.

10. The method of claim 9, wherein the fibers and surfactant are combined with the non-aqueous component prior to combination with the aqueous component.

11. The method of claim 1, including the further step of combining a particulate material with the emulsion.

12. The method of claim 11, wherein said particulate material is sand or ceramic particles.

13. The method of claim 1, wherein said non-aqeous component, aqueous component, surfactant and fibers are combined in a continuous manner.

14. The method of claim 1, wherein said non-aqueous component, aqueous component, surfactant and fibers are combined in a batch-process manner.

15. The method of claim 1, including the further step (f) of pumping said emulsion into a wellbore.

16. The method of claim 1, wherein said non-aqueous component, aqueous component, surfactant and fibers are combined in a well bore.

17. The method of claim 1 wherein said non-aqueous component, aqueous component, surfactant and fibers are agitated in a wellbore.

18. The method of claim 1, wherein said aqueous component and said non-aqueous component are combined prior to combination with the surfactant and fibers.

19. An emulsion having an internal phase and an external phase, comprising:
(a) an aqueous component;
(b) a non-aqueous component;
(c) a surfactant; and
(d) fibers.

20. The emulsion of claim 19, wherein said emulsion is an oil-in-water emulsion.

21. The emulsion of claim 19, wherein said emulsion is a water-in-oil emulsion.

22. The emulsion of claim 19, wherein said external phase is crosslinked.

23. The emulsion of claim 19, wherein the external phase contains a viscosity-increasing agent.

24. The emulsion of claim 23, wherein said viscosity increasing agent is a soluble polymer.

25. The emulsion of claim 24, wherein said soluble polymer is selected from the group consisting of: guar, modified guar, polyacrylamide polymer, polyacrylamide copolymers, hydroxyl ethyl cellulose or xanthan.

26. The emulsion of claim 19, wherein said non-aqueous component is selected from the group consisting of: diesel, kerosene, mineral oil, vegetable oil or crude oil.

27. The emulsion of claim 19, wherein said aqueous component comprises salt.

28. The emulsion of claim 19, wherein said aqueous component comprises a polymer.

29. The emulsion of claim 19, further comprising a reactive species.

30. The emulsion of claim 29, wherein said reactive species is a crosslinking agent.

31. The emulsion of claim 30, wherein said crosslinking agent is boric acid, sodium borate, a titanium complex, a zirconium complex or a dialdehyde.

32. The emulsion of claim 29, wherein said reactive species is a cement-retarding agent.

33. The emulsion of claim 29, wherein said reactive species is a pH modifier.

34. The emulsion of claim 29, wherein said pH modifier is a buffer.

35. An emulsion comprising:
(a) about 0.1-2% of fibers (by weight);
(b) about 1-30% by volume of an aqueous component;
(c) about 70-98.7% by volume of a non-aqueous component; and
(d) about 0.2-2% surfactant.

36. An emulsion comprising:
(a) about 0.1-2% of fibers (by weight);
(b) about 1-30% by volume of an oil (non-aqueous) continuous phase;
(c) about 70-98.7% by volume of an aqueous) phase; and
(d) about 0.2-2% surfactant.

37. A heterogeneous structured fluid comprising:
(a) a bridging fiber;
(b) substantially spherical non-aqueous droplets; and
(c) a continuous minimal non-aqueous phase.
